(19) United States
(12) Patent Application Publication
(10) Pub. No.: US 2015/0010479 A1
(43) Pub. Date: Jan. 8, 2015

(54) TREATMENT OF MICROBIAL INFECTIONS

(71) Applicant: SINCLAIR PHARMACEUTICALS LIMITED, CHESTER (GB)

(72) Inventor: Simon Youlton, Chester (GB)

(21) Appl. No.: 14/375,578

(22) PCT Filed: Mar. 20, 2013
(86) PCT No.: PCT/GB2013/050728

§ 371 Date: Jul. 30, 2014

(30) Foreign Application Priority Data
Mar. 20, 2012 (GB) 1204864.1
Mar. 21, 2012 (GB) 1204970.6

(51) Int. Cl. A61K 31/5375; A61Q 11/00; A61K 8/49; A61K 31/4425

(52) U.S. Cl. CPC A61K 31/5375; A61K 31/4425; A61Q 11/00; A61K 8/49; A61K 8/4926; A61K 2800/92
USPC 424/49; 514/238.8

(57) ABSTRACT

The present invention relates to the use of compositions in the prevention and/or treatment of infections by a number of microbial species associated with the pathological colonisation of wounds and periodontal lesions. The compositions of the invention comprise a morpholino compound in combination with a cetylpyridinium salt, which have been found by the present inventors to exert either a potentiating or synergistic antimicrobial effect on a range of microbes associated with the above pathological activity. Accordingly, the present invention provides novel uses of such compositions in treating or preventing infections or conditions in wounds or periodontal lesions, caused by the microbial species, in addition to methods for treating or preventing such disorders.

### FIELD OF THE INVENTION

The present invention relates to compositions useful in the prevention and/or treatment of microbial infection.

### BACKGROUND

The living body may be infected by a wide variety of microbes. The mucosa and the surface of the skin are particularly at risk of infection given their exposure to the external environment. When the natural barrier provided by the mucosa and the skin is broken by injury or surgery, microbes can spread throughout the layers of the skin and mucosa, which can lead to infection of the wound site. This is particularly the case following invasive surgery. In this instance, the surgical wound may become infected by microbes naturally present in the environment, on the surface of the skin or mucosa, or on the surgical instruments used in the procedure. The infection of wounds causes the healing process to be extended and, in some cases, where wounds are infected, they may fail to heal.

Morpholino compounds are known in the art to have low antibacterial activity. These compounds alone are particularly effective at inhibiting the formation of biofilms, and have been used in this context in WO 06/082393 in compositions to be applied to abiotic surfaces. Synergistic combinations of morpholino compounds with other agents have also been used, for example in the context of dental care, where the inclusion of chelating agents has been found to be effective against the growth of oral plaque bacteria (U.S. Pat. No. 5,147,632).

The antimicrobial activity of quaternary ammonium compounds, such as cetylpyridinium salts, is also known in the art. JP 2002256155 discloses the antimicrobial activity of cetylpyridinium salts alone against oral plaque bacteria, whereas JP 8151325 and US 2003/0211053 disclose synergistic antimicrobial combinations of the salts with lysine derivatives and proteolytic enzymes respectively, with the latter formulated for the treatment of plaque and halitosis. Furthermore, WO 92/08442 describes compositions comprising morpholino compounds in combination with a range of antimicrobial agents (including cetylpyridinium salts), for use in treating or preventing plaque and gingivitis in the oral cavity; wherein such compositions are demonstrated as having inhibitory actvity on the growth of two bacterial species associated with dental plaque (Streptococcus mutans and Bacteriodes melaninogenicus).

Accordingly, there is an overwhelming bias in the prior art towards the use of cetylpyridinium salts and morpholino compounds in the treatment of mild disorders of the oral cavity, such as halitosis and plaque. The nature of microbial colonisation in different pathologies is dependent on a number of factors, including the associated microbes, and environmental factors such as temperature, nutrient and fluid levels, and pH. Dental plaque comprises largely gram-positive cocci bacteria in the form of a polymicrobial biofilm on the tooth surface. With regard to environmental factors, in species such as S. mutans, the presence of sucrose in the oral cavity has been found to enhance initial bacterial adherence and subsequent colonisation.

The nature of microbial infection in wounds and periodontitis is distinct from that of plaque, by virtue of the differences in associated microbial species, physiological conditions, and severity of the conditions. James et al. (Wound Rep Reg. 2008. 16. 37-44) have argued the former two conditions as being analogous in their pathological mechanism, due to the role of diverse microbial communities acting in consort over time to cause a chronic infection. Severe adult periodontitis is largely characterised by the presence of the gram-negative anaerobe Porphyromonas gingivalis in periodontal lesions, whereas infected wounds may be colonised by a variety of microbes, including bacteria from the Staphylococcus and Enterococcus genera. There is consequently a need to provide compositions which are effective in the treatment of the above chronic infections, which must have efficacious antimicrobial properties against the species implicated in these disorders.

### SUMMARY OF THE INVENTION

The present invention is based on the finding that compositions comprising compounds of formula (I) and a cetylpyridinium salt possess synergistically improved abilities to treat and/or prevent infections caused by a range of microbial species, or that compounds of formula (I) act to potentiate the anti-microbial effects of the cetylpyridinium salt against said species. These microbial species are known in the art to be associated with disorders such as chronic infections of skin or mucosal wounds and periodontal lesions. Therefore, in contrast to the above-mentioned prior art, which shows efficacy against bacteria associated with plaque or gingivitis, the present invention provides for the treatment or prevention of infections in wounds of the skin or oral mucosa, including periodontal lesions.

According to a first aspect of the invention, the present invention provides a composition comprising a morpholino compound in combination with a cetylpyridinium salt, the morpholino compound having the general formula (I)

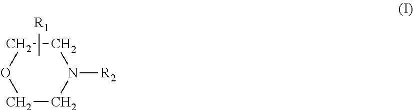

wherein R1 is a straight or branched alkyl group containing 8 to 16 carbon atoms at the 2- or 3-position of the morpholino ring, and R2 is a straight or branched alkyl group containing 2 to 10 carbon atoms, substituted with a hydroxyl group except in the alpha-position, or pharmaceutically acceptable salts thereof for use in the treatment or prevention of an infection or a condition in a patient caused by a microbe selected from the group consisting of Staphylococcus spp, Actinobacillus spp, Campylobacter spp, Fusobacterium spp, Eikenella spp, Cornybacterium spp, Finegoldia spp, Anaerococcus spp, Serratia spp, Escherichia spp, Enterococcus spp, Candida spp, Prevotella spp, Cornybacterium spp, Peptoniphilus spp, Finegoldia spp, Peptostreptococcus spp, Actinomyces spp, Pseudomonas spp and Porphyromonas spp and combinations thereof, wherein the infection is in a wound of the skin or mucosa of a patient, or the condition is periodontitis

According to a second aspect of the invention, the present invention provides a composition as defined above for use in treating or preventing a post-operative infection in or on the skin or mucosa of a patient.

According to a third aspect of the invention, the present invention provides a composition as defined above for use in pre-operatively reducing the amount of infection-causing microbes present in or on the skin or mucosa of a patient.

According to a fourth aspect of the invention, the present invention provides a method of treating or preventing an infection or condition in a patient, wherein said infection or condition is caused by a microbe selected from the group consisting of Staphylococcus spp, Actinobacillus spp, Campylobacter spp, Fusobacterium spp, Eikenella spp, Cornybacterium spp, Finegoldia spp, Anaerococcus spp, Serratia spp, Escherichia spp, Enterococcus spp, Candida spp, Prevotella spp, Cornybacterium spp, Peptoniphilus spp, Finegoldia spp, Peptostreptococcus spp, Actinomyces spp, Pseudomonas spp and Porphyromonas spp and combinations thereof, comprising applying to the patient a composition comprising a morpholino compound in combination with a cetylpyridinium salt, the morpholino compound having the general formula (I)

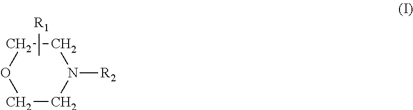

wherein R1 is a straight or branched alkyl group containing 8 to 16 carbon atoms at the 2- or 3-position of the morpholino ring, and R2 is a straight or branched alkyl group containing 2 to 10 carbon atoms, substituted with a hydroxyl group except in the alpha-position, or pharmaceutically acceptable salts thereof, wherein said infection is in a wound of the skin or mucosa of a patient, or the condition is periodontitis.

### DESCRIPTION OF THE FIGURES

The invention is described with reference to the accompanying figure, wherein:

FIG. 1 shows Fractional inhibitory concentration (FIC) index value ranges used to classify whether a compound potentiates or synergistically improves the antimicrobial effect of a given compound.

### DETAILED DESCRIPTION OF THE INVENTION

The present invention provides novel uses of compositions comprising a compound of formula (I) and a cetylpyridinium salt. The above components have been found to display synergistic antimicrobial activity against microbes which cause infection or conditions in or on the skin or mucosa of a patient, thus indicating their suitability for use in treating such disorders.

The morpholino alcohols can be prepared by several processes as described in U.S. Pat. No. 4,636,382 such as:

(a) by alkylating a morpholino derivative having the formula

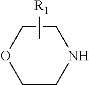

wherein R₁ is as defined above; with an alkylating agent of the formula

R₂X

wherein R₂ is as defined above and X is halogen or an organic sulfonic ester, or wherein X together with a hydroxyl group present in R₂ is a reactive oxide;

(b) by ring closure of a compound having the general formula

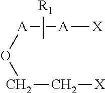

wherein R₁ is as defined above, X is halogen or an organic sulfonic ester and A represents CH₂ groups, one CH₂ group being substituted with the group R₁; with an amino alkanol of the general formula

NH₂R₂

wherein R₂ is as defined above;

(c) by reducing a mono- or di-oxo substituted morpholine having the general formula

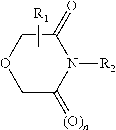

wherein R₂ is as defined above, n is 0 or 1, and R₁ is as defined above and is at the 2-position when n is 1 and at the 2- or 3-position when n is 0; or

(d) by starting from a morpholino compound having the general formula

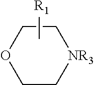

wherein R₁ is as defined above and R₃ is a straight or branched alkyl group containing a group transformable to OH or CH₂OH; especially
(d1) by converting a compound of the formula VIII, wherein the group in R₃ transformable to OH is halogen, NHAc, OAc, O-alkyl, O—CH₂C₆H₅; or
(d2) by converting a compound of the formula VIII, wherein the group in R₃ transformable to CH₂OH is —COOC₂H₅, —CN, —CHO; or
(d3) R₃ represents —CO(CH₂)ₙ—COOC₂H₅ (n=0-8).

The morpholino compound may also be prepared by the method as described in WO 2007/057681 and WO 2007/091009.

The morpholino compound used in this invention can be used in its free base form or as a pharmaceutically-acceptable salt. Examples of pharmaceutically-acceptable salts are the salts of acids such as acetic acid, phosphoric acid, boric acid, hydrochloric acid, maleic acid, benzoic acid, citric acid, maleic acid, oxalic acid, tartaric acid, succinic acid, glutaric acid, gentisic acid, valeric acid, gallic acid, beta-resorcyclic acid, acetyl salicylic acid, salicylic acid, perchloric acid, barbituric acid, sulfanilic acid, phytic acid, p-nitro benzoic acid, stearic acid, palmitic acid, oleic acid, myristic acid, lauric acid; however others known to the skilled person may be used. The most preferred salts are those of hydrochloric acid.

The most preferred morpholino compound used in the present invention is delmopinol, which has the non-proprietory name (INN) of (3-(4-propylheptyl)-4-(2-hydroxyethyl)morpholine) with CAS No. 79874. Equally most preferred is the hydrochloride salt of delmopinol.

The claimed morpholino compounds are known per se, as described in U.S. Pat. No. 4,894,221 and U.S. Pat. No. 5,082,653, which are incorporated herein by reference.

The antimicrobial agent used in the compositions of the present invention is a cetylpyridinium salt. The present inventors have found that the combination of a compound of formula (I) with a cetylpyridinium salt acts synergistically to treat or prevent microbial infection in or on the skin or mucosa, or related conditions in a patient. The cetylpyridinium salt may be used in the form of a pharmaceutically-acceptable salts of acids such as acetic acid, phosphoric acid, boric acid, citric acid, malic acid, oxalic acid, tartaric acid, succinic acid, gluratic acid, gentisic acid, valeric acid, gallic acid, beta-resorcyclic acid, acetylsalicyclic acid, salicyclic acid, perchloric acid, barbituric acid, sulfonic acid, phytic acid p-nitro benzoic acid, stearic acid, palmitic acid, oleic acid, myristic acid, lauric acid and others known to the skilled person. Preferably the cetylpyridinium salt is a halide salt and more preferably, the cetylpyridinium salt is cetylpyridinium chloride.

In the composition of the present invention, the morpholino compound is present preferably in an amount ranging from about 0.005% to about 20.0% by total weight of the composition, preferably from about 0.005% to 5%, more preferably from about 0.01% to about 1.0%, and most preferably from about 0.05% to about 0.2%. The amount of cetylpyridinium salt in the composition preferably ranges from about 0.001% to about 5.0% by total weight of the composition, more preferably from about 0.002% to about 2.0%; and most preferably from about 0.005% to about 1.0%. In one embodiment, the morpholino compound and cetylpyridinium salt are provided in a 1:1 weight ratio. Preferably, in said embodiment, the morpholino compound is provided at 0.1% and the cetypyridinium salt is provided at 0.1% of the total weight of the composition.

Infections or conditions caused by microbes can be reduced or prevented using the compositions of the present invention; wherein the antimicrobial action of the components of said compositions is synergistic compared to that of known compositions comprising a compound of formula (I) and other antimicrobial agents. Accordingly, compounds of formula (I) act to potentiate the antimicrobial effect of the cetylpyridinium salt. The term “microbe” includes bacteria, fungi and yeast. Microbial species against which compositions of the present invention may be effective may be gram-positive, gram-negative, aerobic or anaerobic.

The microbial species against which compositions of the present invention are effective are selected from the group consisting of Staphylococcus spp, Actinobacillus spp, Campylobacter spp, Fusobacterium spp, Eikenella spp, Cornybacterium spp, Finegoldia spp, Anaerococcus spp, Serratia spp, Escherichia spp, Enterococcus spp, Candida spp, Prevotella spp, Cornybacterium spp, Peptoniphilus spp, Finegoldia spp, Peptostreptococcus spp, Actinomyces spp, Pseudomonas spp and Porphyromonas spp and combinations thereof. More preferably, the microbe may be selected from the group consisting of P. gingivalis, Prv. nigrescens, S. aureus E. faecalis, E. coli, C. albicans, C. rectus, F. nucleatum, B. forsythus, E. corrodens and combinations thereof.

As used herein, the nomenclature “spp”, following a given microbial genus, refers to any microbial species belonging to said genus, as is common practice in the art.

In the context of the present invention, an infection or condition in a patient may be considered as being “caused” by a given microbe or combination of microbes, when the absence of said microbe or combination would be understood by the skilled person to ameliorate the infection or condition, when compared with the presence of said microbe or combination. Accordingly, the skilled person would understand the microbe or combination of microbes to exert a pathological effect in the context of the infection or condition. Preferably, said microbe or combination would be understood by the skilled person to provide greater amelioration of the infection or condition if removed, than all other known pathological microbes (in the context of the infection or condition) if removed. More preferably, said microbe or combination would be understood by the skilled person to provide greater amelioration of the infection or condition if removed, than all other known pathological agents (in the context of the infection or condition) if removed. Said microbe or combination does not have to be the sole pathological agent in the infection or condition, although this is most preferable.

The microbes against which the compositions of the present invention are particularly effective are those which infect the skin or mucosa such as the lining of the oral cavity, nasal passages, lips, eyelids, ears, genitalia, and anus, or those which may be found on the surface of the skin. In particular, the components of compositions of the present invention are synergistically effective against microbes which cause infections in wounds in the skin and or mucosa. Preferably, compositions of the present invention are effective at treating or preventing infections in wounds of the non-oral mucosa. The compositions of the present invention may therefore be used to treat or prevent infections in such wounds.

The microbes against which the compositions of the present invention are effective may be involved in the pathogenesis of periodontitis. Thus, compositions of the present invention may be used to treat or prevent conditions such as periodontitis.

In the context of the present invention, a wound is understood to mean any breach of the surface of the skin or mucosa which may be caused by physical injury, such as caused when the skin or mucosa is physically penetrated or damaged by a burn or caused by infection or disease. The wound may be superficial or may be deeper, penetrating several layers of the mucosa or skin. Preferably the wound is in the skin, or may be caused by periodontitis (a periodontal lesion).

The infection of the wound in the skin or mucosa may include the microbial genera Staphylococcus spp, Enterococcus spp, Prevotella spp, Cornybacterium spp, Actinomyces spp, Pseudomonas spp, Porphyromonas spp, Finegoldia spp, Peptostreptococcus spp, Anaerococcus spp, Serratia spp or combinations thereof; particularly the species S. aureus, E. faecalis, E. coli, C. albicans or combinations thereof.

In certain embodiments, compositions of the present invention may be effective at treating or preventing periodontitis. Periodontitis is a condition in which the gums are pulled away from the teeth to form pockets that are open to infection (periodontal lesions). The condition can lead to bone loss from around the tooth root and jaw. Microbes which are implicated in causing periodontitis include the genera Prevotella. spp, Actinomyces spp, Porphyromonas spp, Candida spp or combinations thereof, particularly the species P. gingivalis and C. albicans or combinations thereof.

The compositions of the present invention may be used pre-operatively to treat the skin or mucosa at the site at which an incision is to be made. Preferably, the area of the body that is contacted with the composition of the present invention comprises the site of contact between the body and the surgical instrument (or a portion of said site), and/or the immediate surrounding area.

The healing of wounds caused by injury or surgical incision may be limited if the wound is infected by a microbe. Compositions of the present invention may help promote wound healing by preventing or treating the microbial infection at the site of the wound. Preferably, the wound is treated with the composition of the present invention post-injury or post-operatively. Preferably the wound is in the oral cavity or the skin. Compositions of the present invention may be used pre-operatively to sterilise the surface of the body in which an incision is to be made and/or may be used post-operatively to prevent infection or to treat an infection at the incision site.

More preferably, the infection is caused by contacting the body with a medical device. The compositions of the present invention may be used to treat an abiotic surface, such as that of a medical device, before, during or after, preferably immediately after, initial contact between a medical device and the body. A medical device may include a surgical instrument.

In a preferred embodiment, a composition of the present invention is used to prevent or reduce infection caused by planktonic microbes. As used herein, the term planktonic refers to microbes that are not attached to a surface; planktonic microbes are free floating. These microbes are not part of a biofilm, but may be the same species as microbes comprising a biofilm. A composition of the present invention can be used to act on such microbes before they attach to the human body.

The compound of formula (I) and the cetylpyridinium salt comprising the composition of the invention may be applied to a given site simultaneously, such that the composition is formed prior to administration, or sequentially (in any order), such that the composition is formed upon application of the second (later applied) component.

The composition of the invention may be in the form of a medicament comprising pharmaceutically acceptable excipients that are well known in the art. The composition of the invention may be formulated to additionally comprise sweeteners, flavourants and/or colorants particularly when the composition is intended for oral use. Natural sweeteners, flavourants and/or colorants are known in the art.

The composition of the invention may be delivered to the skin or mucosa in any suitable form or amount that achieves the desired effect. Preferably, the composition is formulated as a gel, foam, cream, emollient, spray, wipe mouthwash, toothpaste, chew or gum. The compositions of the present invention may be applied to the oral mucosa by various methods including, but not limited to brushing, spraying, painting or rinsing of the oral cavity with the composition. The compositions of the present invention may be applied to the skin by topical application, for example through the use of a dressing comprising the composition, which contacts the skin; preferably wherein the dressing is suitable for contacting a wound, for example wherein the dressing is a bandage, gauze, plaster, hydrogel or other medical dressing. Alternatively, the composition may be applied to the skin as an aqueous wash, for example by way of a wound irrigation wash comprising the composition. The composition may also be applied to the skin as a gel or spray.

The following non-limiting Examples illustrate the invention.

### EXAMPLES

The effect on the antimicrobial ability of compositions comprising delmopinol with or without cetylpyridinium chloride (CPC) was studied. Manufacturing details of CPC are provided below:

| Com- | | | | | Sol- |
|---|---|---|---|---|---|
| pound | CAS | Supplier | Cat. No. | Purity | vent |
| CPC | 123 - 03 - 5 | MP | 0219017725 | 99.8% | dH₂O |
| | | Biomedicals | | | |

The compositions of the invention were tested as a solution having the following base composition to which delmopinol and/or CPC was added:

Broth A was prepared, which comprises a base formulation intended for use in oral care applications but without delmopinol or CPC (below) were produced separately then mixed and stirred thoroughly before the final pH was adjusted to 5.6 to 5.7 using 1 M NaOH/HCl as required. The final mixture was then sterilized by filtration using a 0.45 μM PES filter (Nalgene, 169-0045).

Bacterial preparations were prepared using freshly grown cultures on solid media. Several loop-fulls of culture were transferred to 10-20 ml of the appropriate broth and vortexed gently until a fine suspension was produced. The initial OD₆₀₀ nm of the cell suspensions was measured and adjusted to 0.2 to give a colony count of 1×10⁸ cfu/ml. Final inocula were prepared by diluting the adjusted cell suspensions in appropriate broth as detailed below:

| Microbe | Dilution |
|---|---|
| C. albicans NCTC 1363 | 1 in 1000 |
| Ent. faecalis ATCC 29212 | 1 in 100 |
| E. coli ATCC 25922 | 1 in 100 |
| P. gingivalis NCTC 11834 | 1 in 20 |
| Prv. nigrescens ATCC 33563 | 1 in 20 |
| S. aureus ATCC 29213 | 1 in 200 |

Antimicrobial Activity of Delmopinol or CPC Alone

The antimicrobial activity of CPC and delmopinol was determined in both standard media and Broth A. 195 μl of normal broth (standard assay) or broth A described above was added to a 96 well plate. The broths were added to wells in columns 1, 11 (negative control, broth only) and 12 (positive control).

100 μl of normal broth (standard assay) or broth A was added to wells in columns 2-10. 5 μl of either a CPC or delmopinol stock (prepared in a suitable solvent to 80× the desired final concentration) was then added to wells in column 1 and mixed thoroughly (minimum three replicates). Doubling dilutions were prepared by transferring 100 μl of the mixture from column 1 into column 2, then column 2 to column 3 and so on until column 10.

After mixing in column 10, 100 μl was discarded. 5 μl of the appropriate solvent was added to wells in column 12, mixed and 100 μl discarded.

Finally, 100 μl of inoculum (prepared in either normal broth or 2× broth A) was added to the wells in columns 1-10 and 12 and the plates were incubated as described above.

Following the incubation period, plates were examined for growth by eye. The first well of each row that contained no growth was marked at the minimum inhibitory concentration (MIC).

5 μl samples from all wells exhibiting no growth and the first well exhibiting growth were transferred to an appropriate agar plate, allowed to soak into the agar and then incubated. Following this incubation period plates were examined and the lowest concentration of test compound where no growth was recorded as the minimum bactericidal concentration (MBC).

Antimicrobial Activity of Delmopinol and CPC in Combination

Assays were set up as described above except that:

A delmopinol stock solution (in dH₂O) prepared to 200× the desired final concentration (this would be ¼ of its recorded MIC alone) was diluted 1 in 100 into the inocula and then 100 μl immediately added to all wells in columns 1-10 and 12.

Rows A-C record MIC/MBC for the test compound alone and rows F-H record MIC/MBC of test the compound in combination with a ¼ MIC of delmopinol.

The mode of interaction between CPC and delmopinol was interpreted by converting MIC data into fractional inhibitory concentration index (FICi) score using the following formula:

FIC_{(CPC)}=MIC of CPC with delmopinol/MIC of CPC alone

FIC_{(Delmopinol)} is fixed at 0.25 (i.e. ¼ MIC)

FICi_{(CPC)}=FIC+FIC_{(Delmopinol)}

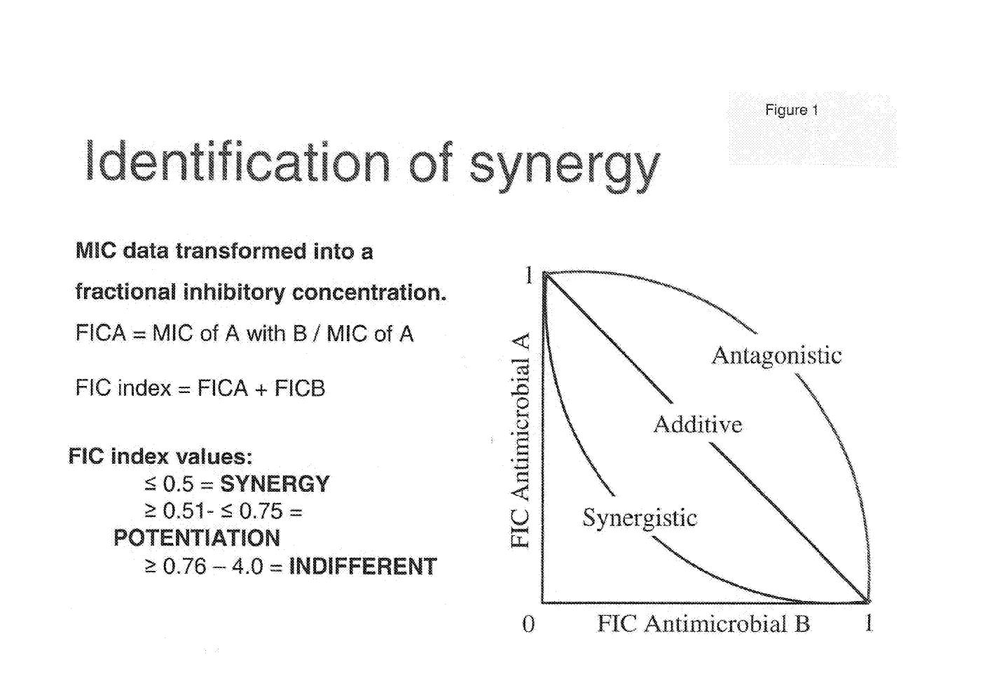

The FICi was then interpreted using the following parameters (this is depicted in FIG. 1):

Synergy=FICi≦0.5

Potentiation=FICi>0.5-0.75

Indifferent=FICi>0.75-4

Antagonism=FICi>4

A fractional bactericidal concentration index (FBCi) was calculated and interpreted in the same manner but using MBC data. The FBC_{(Delmopinol)}, although fixed, would be less than or equal to 0.25 depending on what fraction of ¼ MIC concentration is relative to its MBC alone.

Antimicrobial Activity of CPC and Delmopinol Combinations (Chequerboard Assay)

Separate CPC and delmopinol stocks were prepared in dH₂O to 80× the desired final concentration and then a series of two fold dilutions were prepared in dH₂O.

190 μl of normal broth (standard assays) or broth A was added to wells of a 96 well plate. 5 μl of each dilution of CPC was added to the appropriate wells of the 96 well plate. 5 μl of each dilution of delmopinol was added to the appropriate wells of the 96 wells plate. The contents of each well were mixed thoroughly and then 100 μl volume was discarded.

100 μl of inocula was added to all wells in columns 1-10 and 12. Plates were then incubated as described above. Following the incubation period, plates were examined for growth by eye and all wells were recorded for the presence or absence of growth (used for FIC determination). 5 μl samples of all wells were transferred to an appropriate agar plate, allowed to soak in agar and then incubated. Following incubation the plates were examined and growth/no growth recorded for every well sample (used for FBC determination).

FIC_{(CPC)}=MIC of CPC with delmopinol/MIC of CPC alone

FIC_{(Delmopinol)}=MIC of delmopinol with CPC/MIC of delmopinol alone

FICi=FIC_{(CPC)}+FIC_{(Delmopinol)}

FBCi were calculated in the same manner but using MBC data.

The FICi and FBCi were interpreted using the same parameters as set out above.

Antimicrobial Activity of Decapinol and CPC in Combination

Delmopinol is commercially available as a mouthwash and is sold under the trademark Decapinol. Decapinol (for testing delmopinol plus CPC) was reformulated to contain 1:1 ratio of CPC to delmopinol i.e. 0.1% each. Decapinol without delmopinol (for testing CPC alone) was also reformulated.

Further formulations of complete Decapinol with CPC and Decapinol without delmopinol but with CPC were made to give a ratio of DEL:CPC of 50:1.

195 μl of Broth A was added to wells in column 11 (negative control) of a 96 well round bottom plate. 100 μl of Broth A was added to wells in column 2-10 and 12 (positive control).

200 μl of the adjusted ratio formulations (Decapinol+/−delmopinol), 1:1 ratio formulations (Decapinol+/−delmopinol and diluted 1 in 16 in Broth A) or complete Decapinol (for testing delmopinol alone) were added to wells in column 1.

Doubling dilutions were prepared by transferring 100 μl of the mixture from column 1 into column 2, then column 2 to column 3 and so on until column 10. 100 μl of inocula (prepared in 2× broth) was added to all wells in columns 1-10 and 12. Plates were then incubated as described in Table 1.

Following the incubation period, plates were examined for growth by eye. The first well of each row that contained new growth was marked as the minimum. The first well of each row that contained no growth was marked as the MIC.

5 μl samples from all wells exhibiting no growth and the first well exhibiting growth were transferred to an appropriate agar plate, allowed to soak in agar and then incubated. Plates were then examined and the lowest concentration of test compound where no growth occurred was recorded as the MBC.

The mode of interaction between CPC and delmopinol was defined by converting MIC and MBC data into FICi and FBCi and interpreted using the parameters described above.

Results and Discussion

Tables 1-12 show the antimicrobial mode of action (potentiation or synergy) of delmopinol and cetylpyridinium chloride (CPC) on Porphyromonas gingivalis (Tables 1 and 2), Prevotella nigrescence (Tables 3 and 4), Staphylococcus aureus (Tables 5 and 6), Entercoccus faecalis (Tables 7 and 8), Escherichia coli (Tables 9 and 10), Candida albicans (Tables 11 and 12). The results are summarised in Table 13.

TABLE 1
P. gingivalis

| | Activity alone (ug/ml) | | | | Combination with delmopinol (FIC) | | | | Evaluation in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 0.49 | 0.49 | 0.49 | 0.49 | 0.62p | 0.75p | 1.5 | NEP | 1.01 | 1.01 | 0.75p | 0.75p |
| Delmopinol | 1.95 | 31.25 | 31.25 | 31.25 | | | | | | | | |

*No delmopinol or sodium benzoate present
\hat{ }Ratios: 50:1 (Del:CPC)
p= potentiation

TABLE 2
P. gingivalis

| | Activity alone (uM) | | | | Combination with delmopinol (FIC) | | | | Evalution in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 1.4 | 1.4 | 1.4 | 1.4 | 0.62p | 0.75p | 1.5 | NEP | 1.01 | 1.01 | 0.75p | 0.75p |
| Delmopinol | 10 | 120 | 1.20 | 120 | | | | | | | | |

*No delmopinol or sodium benzoate present
\hat{ }Ratios: 50:1 (Del:CPC)
p= potentiation

TABLE 3
Prv. nigrescence

| | Activity alone (ug/ml) | | | | Combination with delmopinol (FIC) | | | | Evaluation in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 0.06 | 0.49 | 0.25 | 0.49 | 0.75p | 0.79 | 0.5s | 0.53p | 1 | 1 | 0.38s | 1.5 |
| Delmopinol | 62.5 | 62.5 | 31.25 | 125 | | | | | | | | |

*No delmopinol or sodium benzoate present
\hat{ }Ratios: 50:1 (Del:CPC)
p= potentiation
s= synergy

TABLE 4
Prv. nigrescence

| | Activity alone (uM) | | | | Combination with delmopinol (FIC) | | | | Evaluation in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 0.2 | 1.4 | 0.7 | 1.4 | 0.75p | 0.79 | 0.5s | 0.53p | 1 | 1 | 0.38s | 1.5 |
| Delmopinol | 230 | 230 | 120 | 460 | | | | | | | | |

*No delmopinol or sodium benzoate present
\hat{ }Ratios: 50:1 (Del:CPC)
p= potentiation
s= synergy

TABLE 5
S. aureus

| | Activity alone (ug/ml) | | | | Combination with delmopinol (FIC)# | | | | Evaluation in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 3.9 | 7.8 | 0.49 | 1.95 | 0.38s | 0.63p | 0.38s | 0.75p | 1 | 0.5s | 0.63p | 0.38s |
| Delmopinol | 125 | 250 | 250 | 250 | | | | | | | | |

*No delmopinol or sodium benzoate present
\hat{ }Ratios: 50:1 (Del:CPC)
p= potentiation
s= synergy
#¼ delmopinol expts (Not full FIC/FBC)

TABLE 6
S. aureus

| | Activity alone (uM) | | | | Combination with delmopinol (FIC)# | | | | Evaluation in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 11.5 | 22.9 | 1.4 | 5.7 | 0.38s | 0.63p | 0.38s | 0.75p | 1 | 0.5p | 0.63p | 0.38s |
| Delmopinol | 460 | 920 | 920 | 920 | | | | | | | | |

*No delmopinol or sodium benzoate present
\hat{ }Ratios: 50:1 (Del:CPC)
p= potentiation
s= synergy
#¼ delmopinol expts (Not full FIC/FBC)

TABLE 7
E. faecalis

| | Activity alone (ug/ml) | | | | Combination with delmopinol (FIC)# | | | | Evaluation in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 1.95 | 15.6 | 0.98 | 3.9 | ND | ND | 1.25 | 1.25 | 1 | 1.02 | 0.75p | 1.5 |
| Delmopinol | 250 | 250 | 250 | 250 | | | | | | | | |

*No delmopinol or sodium benzoate present
\hat{ }Ratios: 50:1 (Del:CPC)
p= potentiation
s= synergy
#¼ delmopinol expts (Not full FIC/FBC)

TABLE 8
E. faecalis

| | Activity alone (uM) | | | | Combination with delmopinol (FIC)# | | | | Evaluation in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 5.7 | 45.9 | 2.9 | 11.5 | ND | ND | 1.25 | 1.25 | 1 | 1.02 | 0.75p | 1.5 |
| Delmopinol | 920 | 920 | 920 | 920 | | | | | | | | |

*No delmopinol or sodium benzoate present
\hat{ }Ratios: 50:1 (Del:CPC)
p= potentiation
s= synergy
#¼ delmopinol expts (Not full FIC/FBC)

TABLE 9
E. coli

| | Activity alone (ug/ml) | | | | Combination with delmopinol (FIC)# | | | | Evaluation in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 15.6 | 31.25 | 15.6 | 62.5 | 0.75p | 1.25 | 0.75p | 1.25 | 1.06 | 1.06 | 1.25 | 1.25 |
| Delmopinol | 250 | 250 | 250 | 250 | | | | | | | | |

*No delmopinol or sodium benzoate present
\hat{ }Ratios: 50:1 (Del:CPC)
p= potentiation
s= synergy
#¼ delmopinol expts (Not full FIC/FBC)

TABLE 10
E. coli

| | Activity alone (uM) | | | | Combination with delmopinol (FIC)# | | | | Evaluation in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 45.9 | 91.9 | 45.9 | 183.8 | 0.75p | 1.25 | 0.75p | 1.25 | 1.06 | 1.06 | 1.25 | 1.25 |
| Delmopinol | 920 | 920 | 920 | 920 | | | | | | | | |

*No delmopinol or sodium benzoate present
\hat{ }Ratios: 50:1 (Del:CPC)
p= potentiation
s= synergy
#¼ delmopinol expts (Not full FIC/FBC)

TABLE 11
C. albicans

| | Activity alone (ug/ml) | | | | Combination with delmopinol (FIC) | | | | Evaluation in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 0.98 | 0.98 | 0.49 | 0.98 | 0.63p | 0.68p | 0.99 | 0.65p | 1 | 2 | 1.25 | 1.25 |
| Delmopinol | 500 | 500 | 120 | 120 | | | | | | | | |

*No delmopinol or sodium benzoate present
\hat{ }Ratios: 50:1 (Del:CPC)
p= potentiation
s= synergy

TABLE 12
C. albicans

| | Activity alone (uM) | | | | Combination with delmopinol (FIC) | | | | Evaluation in Decapinol | | | |
|---|---|---|---|---|---|---|---|---|---|---|---|---|
| | Standard | | Decapinol* base | | Standard | | Decapinol* base | | 1:1 Ratio | | Adjusted ratio\hat{ } | |
| Compound | MIC | MBC | MIC | MBC | FICi | FBCi | FICi | FBCi | FICi | FBCi | FICi | FBCi |
| CPC | 2.9 | 2.9 | 1.4 | 2.9 | 0.63p | 0.68p | 0.99 | 0.65p | 1 | 2 | 1.25 | 1.25 |
| Delmopinol | 1840 | 1840 | 460 | 460 | | | | | | | | |

*No delmopinol or sodium benzoate present
\hat{ }Ratios: 50:1 (Del:CPC)
p= potentiation
s= synergy

TABLE 13
Summary of all synergies detected

| Compound + | | | | | | |
|---|---|---|---|---|---|---|
| delmopinol | P. gingivalis | Prv. Nigrescens | S. aureus | E. faecalis | E. coli | C. albicans |
| CPC | potentiation | synergy | synergy | potentiation | potentiation | potentiation |

As displayed in Table 13, the data from the above tables show that compositions comprising delmopinol and CPC in combination, compared to just delmopinol alone or CPC alone, surprisingly have potentiating or synergistic antimicrobial effects on a range of microbes. The reformulated decapinol test shows that the observed potentiation or synergistic antimicrobial activity is not due to the excipients that are present in Decapinol solution.

Accordingly, compositions of the present invention comprising the combination of a morpoholino compound of Formula (I) and a cetylpyridinium salt display an unexpected advantage at treating or preventing infection caused by a range of microbes associated with the pathological colonisation of wounds and periodontal lesions.

I claim:

1. A composition comprising a morpholino compound in combination with a cetylpyridinium salt, the morpholino compound having the general formula (I) 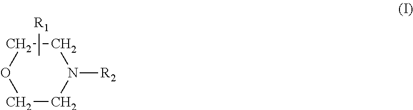
wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxyl group except in the alpha-position, or pharmaceutically acceptable salts thereof, for use in the treatment or prevention of an infection or condition in a patient caused by a microbe selected from the group consisting of Staphylococcus spp, Actinobacillus spp, Campylobacter spp, Fusobacterium spp, Eikenella spp, Cornybacterium spp, Finegoldia spp, Anaerococcus spp, Serratia spp, Escherichia spp, Enterococcus spp, Candida spp, Prevotella spp, Cornybacterium spp, Peptoniphilus spp, Finegoldia spp, Peptostreptococcus spp, Actinomyces spp, Pseudomonas spp and Porphyromonas spp and combinations thereof, wherein the infection is in a wound of the skin or mucosa of a patient, or the condition is periodontitis.

2. The composition according to claim 1, wherein the sum of the carbon atoms in the groups R₁ and R₂ is at least 10, preferably between 10 and 20.

3. The composition according to claim 1, wherein R₂ terminates with the hydroxyl group.

4. The composition according to claim 1, wherein the morpholino compound is 3-(4-propyl-heptyl)-4-(2-hydroxyethyl)morpholine.

5. The composition according to claim 1, wherein the infection or condition is caused by a microbe selected from the group consisting of P. gingivalis, Prv. nigrescens, S. aureus, E. faecalis, E. coli, C. albicans, C. rectus, F. nucleaturn, B. forsythus, E. corrodens and combinations thereof.

6. The composition according to claim 1, wherein the cetylpyridinium salt is a halide salt.

7. The composition according to claim 1, wherein the cetylpyridinium salt is cetylpyridinium chloride.

8. The composition according to claim 1, wherein said composition is in the form of a gel, foam, cream, emollient, spray, wipe, mouthwash, toothpaste, chew, or gum.

9. The composition according to claim 1, for use in treating or preventing a post-operative infection in or on the skin or mucosa of a patient.

10. The composition according to claim 1, for use pre-operatively to reduce the amount of infection causing microbes present in or on the skin or the mucosa of the patient.

11. A method of treating or preventing an infection or condition in a patient comprising applying to the patient a composition comprising a morpholino compound in combination with a cetylpyridinium salt, the morpholino compound having the general formula (I) 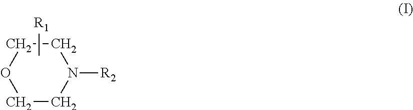
wherein R₁is a straight or branched alkyl group containing 8 to 16 carbon atoms at the 2- or 3-position of the morpholino ring, and R₂ is a straight or branched alkyl group containing 2 to 10 carbon atoms, substituted with a hydroxyl group except in the alpha-position, or pharmaceutically acceptable salts thereof wherein the infection or condition is caused by a microbe selected from the group consisting of Staphylococcus spp, Actinobacillus spp, Campylobacter spp, Fusobacterium spp, Eikenella spp, Cornybacterium spp, Finegoldia spp, Anaerococcus spp, Serratia spp, Escherichia spp, Enterococcus spp, Candida spp, Prevotella spp, Cornybacterium spp, Peptoniphilus spp, Finegoldia spp, Peptostreptococcus spp, Actinomyces spp, Pseudomonas spp and Porphyromonas spp and combinations thereof, wherein the infection is in a wound of the skin or mucosa of a patient, or the condition is periodontitis.

12. The method according to claim 11, wherein the sum of the carbon atoms in the groups R₁ and R₂ is at least 10, preferably between 10 and 20.

13. The method according to claim 11, wherein R₂ terminates with the hydroxyl group.

14. The method according to claim 11, wherein the morpholino compound is 3-(4-propyl-heptyl)-4-(2-hydroxyethyl)morpholine.

15. The method according to claim 11, wherein the infection or condition is caused by a microbe selected from the group consisting of P. gingivalis, Prv. nigrescens, S. aureus E. faecalis, E. coli, C. albicans, C. rectus, F. nucleatum, B. forsythus, E. corrodens and combinations thereof.

16. The method according to claim 11, wherein the cetylpyridinium salt is a halide salt.

17. The method according to claim 11, wherein the cetylpyridinium salt is cetylpyridinium chloride.

18. The method according to claim 11, wherein said composition is in the faun of a gel, foam, cream, emollient, spray, wipe, mouthwash, toothpaste, chew, or gum.

19. The method according to claim 11, wherein the infection is a post-operative infection.

20. The method according to claim 11, wherein the composition is applied to the skin or mucosa pre-operatively to reduce the amount of infection causing microbes present in or on the skin or mucosa of the patient.
